(19) United States
(12) Patent Application Publication
(10) Pub. No.: US 2010/0256208 A1
(43) Pub. Date: Oct. 7, 2010

(54) NOVEL PROCESS

(76) Inventors: Vinayak G. Gore, Maharashtra (IN); Vikas S. Kulkarni, Maharashtra (IN); Sneha R. Wavhal, Maharashtra (IN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/602,922

(22) PCT Filed: Jun. 4, 2008
(86) PCT No.: PCT/GB2008/050409

§ 371 Date: Jun. 1, 2010

(30) Foreign Application Priority Data
Jun. 4, 2007 (IN) 1052/MUM/2007

(51) Int. Cl. A61K 31/4196; C07D 403/10; A61P 25/06

(52) U.S. Cl. 514/383; 548/266.4

(57) ABSTRACT

The present invention relates to a novel process for the preparation of rizatriptan and its pharmaceutically acceptable salts. It provides a novel process for the preparation of highly pure rizatriptan, which can be easily adopted for commercial production with a high degree of consistency in purity and yield. Subsequently the rizatriptan base prepared can be converted into any suitable pharmaceutically acceptable salt, such as the oxalate, succinate or benzoate salt, for dosage form preparation. The present invention also provides a composition comprising rizatriptan useful for the manufacture of a medicament for the treatment or prevention of migraine.

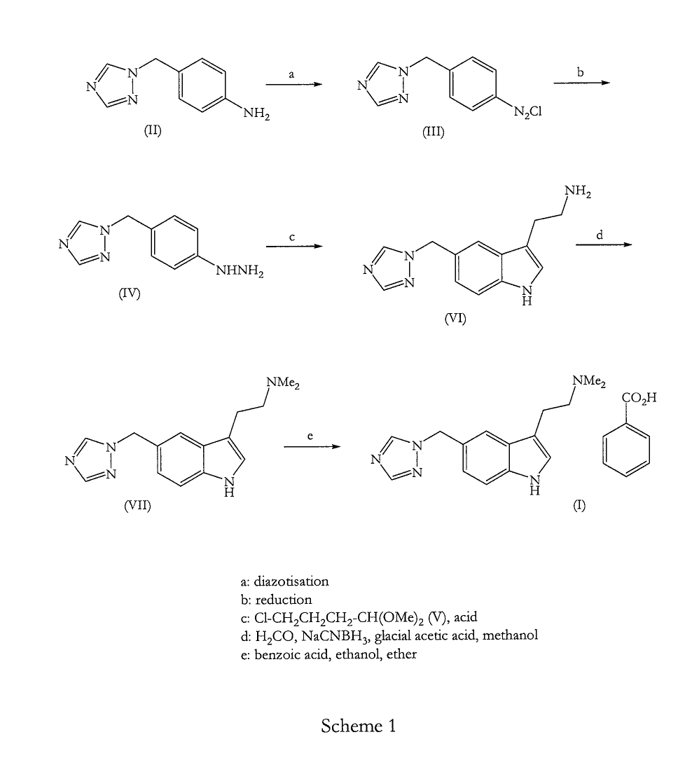

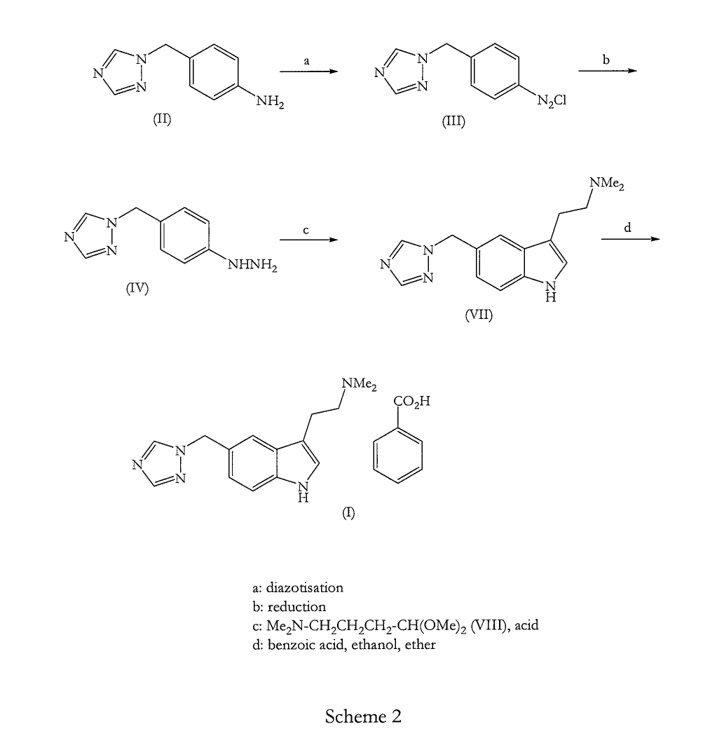

### CROSS-REFERENCE TO RELATED APPLICATION(S)

This application is a Section 371 National Stage Application of International No. PCT/GB2008/050409, filed 4 Jun. 2008 and published as WO 2008/149152 A1 on 11 Dec. 2008, which claims priority from the India Application 1052/mum/2007, filed 4 Jun. 2007, the contents of which are incorporated herein in their entirety for all purposes.

### FIELD OF THE INVENTION

The present invention relates to a novel process for the preparation of rizatriptan and its pharmaceutically acceptable salts. In particular, it relates to a novel process for the preparation of rizatriptan and its pharmaceutically acceptable salts, which is amenable to large-scale production and provides the product with improved yield and purity.

### BACKGROUND OF THE INVENTION

Rizatriptan, chemically named N,N-dimethyl-2-[5-(1,2,4-triazol-1-yl-methyl)-1H-indol-3-yl]ethanamine, is a selective serotonin 5-HT1D receptor agonist and is currently marketed, as the benzoate salt, for the acute treatment of the headache phase of migraine attacks, with or without aura.

Rizatriptan is structurally derived from tryptamine and its therapeutic activity in treating migraine headache may be attributed to its agonist effects at 5-HT1B and 5-HT1D receptors on the extracerebral intracranial blood vessels that are thought to become dilated during an attack and on the trigeminal sensory nerves that innervate them. Activation of these 5-HT1B and 5-HT1D receptors may result in constriction of pain-producing intracranial blood vessels and inhibition of neuropeptide release that leads to decreased inflammation in sensitive tissues and reduced central trigeminal pain signal transmission.

Various patents describe processes for the preparation of rizatriptan base, which can be converted into a desired pharmaceutically acceptable salt. Processes to obtain rizatriptan base and its pharmaceutically acceptable salts disclosed in EP 0497512 and U.S. Pat. No. 5,298,520 are shown in schemes 1 and 2. EP 0497512 and U.S. Pat. No. 5,298,520 describe processes for the preparation of rizatriptan based on a Fischer indole synthesis using the corresponding phenylhydrazine and a protected aldehyde. Accordingly, the phenylhydrazine derivative (IV) is reacted with 4-chloro-butyraldehyde dimethyl acetal (V) to obtain the tryptamine derivative (VI), which on N-methylation gives rizatriptan (VII) (scheme 1). Alternatively, phenylhydrazine derivative (IV) is reacted with 4-N,N-dimethylamino-butyraldehyde dimethyl acetal (VIII) to give rizatriptan (VII) (scheme 2). However, both these processes disclosed in the prior art suffer from disadvantages such as moderate to low yields and the need for column chromatography to isolate rizatriptan in a pure state. Therefore, the processes are not suitable for commercial scale.

A slightly improved process for the preparation of rizatriptan, based on the above mentioned Fischer indole synthesis, is disclosed in EP 0573221, wherein the three steps of diazotisation, reduction and cyclization are carried out in one pot. The process gives rizatriptan in a 45% yield after column chromatography. The main drawback of the process is the need for column chromatography to remove polymeric side products. Therefore the process is not suitable for commercial scale.

WO 2006/137083 describes a modification of the process disclosed in EP 0573221. Accordingly, the indolyzation step is performed at 50-70° C. to minimize the formation of dimeric impurity (XI). Rizatriptan base is isolated as its benzoate salt and the salt is basified to give pure rizatriptan. The pure rizatriptan is finally converted into its benzoate salt. The patent claims that this process produces less dimeric (XI) (less than 3%) and polymeric impurities compared to the earlier processes and that it gives crude rizatriptan in about 60% yield. The process suffers from the disadvantage that for the isolation of rizatriptan distillation of a huge quantity of ethyl acetate is required which, for a kilo scale batch, is time consuming. Moreover the process involves the cumbersome preparation of the benzoate salt followed by neutralization to give rizatriptan base with better purity.

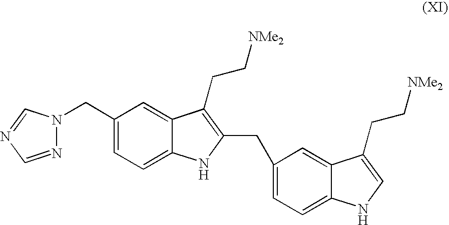

Further alternative processes to obtain rizatriptan base are disclosed in WO 2004/014877, WO 2004/056769, WO 2005/075422, and U.S. Pat. No. 5,567,824.

Consequently there is a need for an improved process for the preparation of rizatriptan and its pharmaceutically acceptable salts, which is amenable to commercial scale and relatively high yielding.

### SUMMARY OF THE INVENTION

The present invention provides a novel process for the preparation of highly pure rizatriptan, which can be easily adopted for commercial production with a high degree of consistency in purity and yield. Subsequently the rizatriptan base prepared by the present invention can be converted into any suitable pharmaceutically acceptable salt, such as the oxalate, succinate or benzoate salts, for dosage form preparation. In addition, the present invention offers a simple work-up procedure with improved yield and purity with minimum contamination with process impurities.

According to a first aspect, the present invention provides a process for the preparation of rizatriptan (VII) or a pharmaceutically acceptable salt thereof, comprising the steps of:
(a) diazotising 4-(1,2,4-triazol-1-yl-methyl)aniline (II) or a salt thereof and then reducing the diazotised product to form hydrazine (IV):

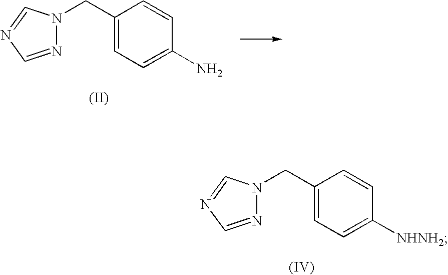

(b) reacting hydrazine (IV), with or without isolation, with 4-N,N-dimethylamino-butyraldehyde or a carbonyl-protected form thereof to form rizatriptan free base (VII):

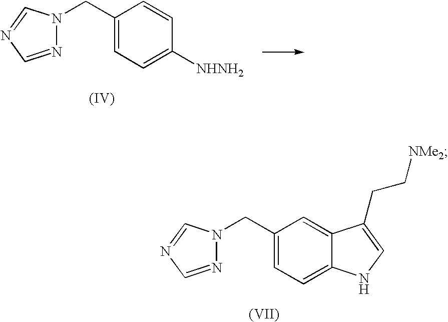

(c) extracting and purifying the rizatriptan free base (VII); and
(d) optionally converting the rizatriptan free base (VII) into a pharmaceutically acceptable salt thereof.

The present invention also provides an improved process for the preparation of rizatriptan benzoate (I):

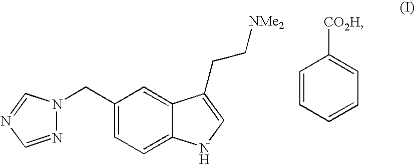

comprising the step of converting purified rizatriptan free base (VII) obtained by the process of the present invention into its benzoate salt by reacting the purified rizatriptan free base (VII) with benzoic acid.

Preferably a salt of aniline (II) is used in step (a), preferably a mineral acid salt or an organic carboxylic acid salt. Preferably the mineral acid salt is the hydrochloric, hydrobromic or sulfuric acid salt. Preferably the organic carboxylic acid salt is the formic, acetic, benzoic or sulfonic acid salt; preferably the sulfonic acid salt is the benzene sulfonic or toluene sulfonic acid salt.

In a most preferred embodiment, 4-(1,2,4-triazol-1-yl-methyl)aniline hydrochloric acid salt (IX) is used in step (a). Preferably the hydrochloric acid salt (IX) is obtained by adding alcoholic HCl (preferably ethanolic HCl) to a solution of 4-(1,2,4-triazol-1-yl-methyl)aniline (II) in the presence of an alcohol (preferably methanol), preferably at a temperature of about 0-10° C., preferably about 5° C.

The diazotisation in step (a) is preferably carried out using an excess of sodium nitrite, preferably up to 7 eq., preferably 2-7 eq., preferably 3-7 eq., preferably 4-7 eq., preferably 5-6 eq., relative to the aniline (II) or the salt thereof. Preferably the diazotisation is carried out in water in the presence of an acid such as hydrochloric acid. Preferably the diazotisation is carried out at a temperature of about −5 to 0° C., preferably about −5° C., preferably for about 30 minutes.

The reduction in step (a) is preferably carried out using sodium sulfite or sodium dithionite, preferably sodium sulfite. Preferably an excess of sodium sulfite (preferably up to 6 eq.) is used. Preferably the reduction is carried out at a temperature of about 25-70° C., preferably about 65-70° C., preferably for about 1-2 hours.

Preferably a carbonyl-protected form of 4-N,N-dimethylamino-butyraldehyde is used in step (b), preferably the dimethyl or diethyl acetal, preferably the diethyl acetal. Preferably step (b) is carried out in the presence of an acid. Preferably the acid is sulfuric acid or hydrochloric acid. Preferably the cyclization reaction of step (b) is carried out at a temperature of about 25-90° C., preferably about 25-70° C., preferably about 25-50° C., more preferably about 25-30° C.

In a preferred embodiment of step (b), hydrochloric acid or sulfuric acid is added to a solution of hydrazine (IV). Preferably the reaction mixture is maintained at a temperature of about 25-70° C., preferably about 65-70° C., preferably for about 1-2 hours, preferably followed by cooling to about 20-30° C. Then, preferably, a carbonyl-protected form of 4-N,N-dimethylamino-butyraldehyde is added to the reaction mixture, preferably whilst maintaining the reaction temperature below about 30° C. Then, preferably, the reaction mixture is maintained at a temperature of about 25-90° C., preferably about 25-70° C., preferably about 25-50° C., more preferably about 25-30° C., preferably for up to about 24 hours.

In a most preferred embodiment, once the 4-N,N-dimethylamino-butyraldehyde or the carbonyl-protected form thereof has been added to the reaction mixture, the reaction temperature is maintained below about 90° C., preferably below about 70° C., preferably below about 50° C., preferably below about 30° C., preferably at about 25-30° C. It has been found that this minimizes the amount of impurities formed, in particular the amount of dimer impurity (XI).

Preferably the extraction and purification of step (c) comprises the steps of: (c1) basifying the reaction mixture (preferably to a pH of about 8.5-9), (c2) extracting crude rizatriptan base into an organic solvent (preferably ethyl acetate), (c3) extracting rizatriptan into an acidic aqueous solution (preferably comprising oxalic, citric or succinic acid, more preferably succinic acid), preferably washing the aqueous solution of acidic rizatriptan with an organic solvent (such as ethyl acetate), (c4) basifying the aqueous solution comprising acidic rizatriptan (preferably to a pH of about 8.5-9), (c5) re-extracting purified rizatriptan base into an organic solvent (preferably ethyl acetate), and (c6) removing the organic solvent.

Preferably the extraction and purification of step (c) comprises the steps of: (c1) basic work-up at a pH of about 8.5-9 (using for example aqueous ammonia), (c2) extraction of crude rizatriptan base into an organic solvent (preferably ethyl acetate), (c3) purification by extraction into an aqueous solution of an organic acid, preferably washing the aqueous solution of acidic rizatriptan with an organic solvent (such as ethyl acetate), (c4) liberation of purified rizatriptan base (for example by basifying the aqueous solution of acidic rizatriptan to a pH of about 8.5-9 with a base such as aqueous sodium hydroxide), (c5) re-extraction of the purified rizatriptan base into an organic solvent (such as ethyl acetate), and (c6) removal of the organic solvent. Organic acids preferably used for the extraction of step (c3) are water soluble organic acids such as oxalic, citric or succinic acid. The acid most preferably used is succinic acid.

Preferably the purified rizatriptan base obtained in step (c) is more than 99.5% pure, preferably more than 99.7%, preferably more than 99.8%, and more preferably more than 99.9% (as measured by HPLC).

Preferably the purified rizatriptan base obtained in step (c) is practically free of dimeric and other impurities. For the purposes of the present invention, “practically free” of dimeric and other impurities means that the purified rizatriptan base contains less than 1% dimeric and other impurities, preferably less than 0.5%, preferably less than 0.1%, and more preferably less than 0.05% (as measured by HPLC).

Preferably the purified rizatriptan base obtained in step (c) is practically free of dimer impurity (XI). For the purposes of the present invention, “practically free” of dimer impurity

(XI) means that the purified rizatriptan base contains less than 1% dimer impurity (XI), preferably less than 0.5%, preferably less than 0.1%, and more preferably less than 0.05% (as measured by HPLC).

Preferably the purified rizatriptan base obtained in step (c) is suitable for conversion into a rizatriptan salt conforming to ICH guidelines and other stringent specifications.

Preferably the purified rizatriptan base in step (c) is obtained on an industrial scale, preferably in batches of 0.5 kg, 1 kg, 5 kg, 10 kg, 50 kg, 100 kg or more.

Preferably the purified rizatriptan base in step (c) is obtained from 4-(1,2,4-triazol-1-yl-methyl)aniline (II) or a salt thereof in a yield of 50%, 60%, 65%, 70%, 75% or more.

Thus the present invention provides a process for the preparation of rizatriptan base with improved yield and purity, which is amenable to large-scale production wherein reaction conditions are reproducible and easily controlled.

Preferred pharmaceutically acceptable salts which may be formed in step (d) are the benzoate, oxalate, succinate, hydrochloride, hydrobromide, acetate, propionate, maleate and fumarate salts. Most preferably, the pharmaceutically acceptable salt formed in step (d) is the benzoate salt. Thus the present invention provides a process for the preparation of rizatriptan benzoate with improved yield and high purity, particularly with respect to levels of the dimer impurity (XI).

Preferably the process of the present invention is carried out without the use of column chromatography. Thus the present invention provides an improved process for the preparation of rizatriptan, avoiding column chromatography purification for isolating rizatriptan, thereby making the process further simpler and adaptable for large-scale production.

A second aspect of the invention comprises rizatriptan or a pharmaceutically acceptable salt thereof, prepared by a process according to the first aspect of the invention.

The second aspect of the present invention also provides rizatriptan, or a pharmaceutically acceptable salt thereof, with more than 99.5% purity, preferably more than 99.7% purity, preferably more than 99.8% purity, and more preferably more than 99.9% purity (as measured by HPLC).

The second aspect further provides rizatriptan, or a pharmaceutically acceptable salt thereof, practically free of dimeric and other impurities. For the purposes of the present invention, “practically free” of dimeric and other impurities means that the purified rizatriptan base contains less than 1% dimeric and other impurities, preferably less than 0.5%, preferably less than 0.1%, and more preferably less than 0.05% (as measured by HPLC).

The second aspect further provides rizatriptan, or a pharmaceutically acceptable salt thereof, practically free of dimer impurity (XI). For the purposes of the present invention, “practically free” of dimer impurity (XI) means that the purified rizatriptan base contains less than 1% dimer impurity (XI), preferably less than 0.5%, preferably less than 0.1%, and more preferably less than 0.05% (as measured by HPLC).

A preferred embodiment of the second aspect of the invention is when the pharmaceutically acceptable salt is the benzoate, oxalate, succinate, hydrochloride, hydrobromide, acetate, propionate, maleate or fumarate salt. Most preferably, the pharmaceutically acceptable salt is the benzoate salt.

A third aspect of the invention is a pharmaceutical composition comprising rizatriptan or a pharmaceutically acceptable salt thereof, according to the second aspect of the invention. A preferred embodiment of the third aspect of the invention is when the pharmaceutically acceptable salt is the benzoate, oxalate, succinate, hydrochloride, hydrobromide, acetate, propionate, maleate or fumarate salt. Most preferably, the pharmaceutically acceptable salt is the benzoate salt.

A fourth aspect of the invention is the use of the compound or composition according to the second and third aspects of the invention for the manufacture of a medicament for the treatment or prevention of migraine.

A fifth aspect of the invention is a method of treating or preventing migraine, comprising administering the compound or composition according to the second and third aspects of the invention to a patient in need thereof Preferably the patient is a mammal, preferably a human.

### BRIEF DESCRIPTION OF THE ACCOMPANYING FIGURES

Schemes 1 and 2 show prior art processes for the preparation of rizatriptan free base (VII) and rizatriptan benzoate (I) as disclosed in EP 0497512 and US 5298520.

### DETAILED DESCRIPTION OF THE INVENTION

In a particularly preferred embodiment, the present invention provides a novel process for the synthesis of rizatriptan and pharmaceutically acceptable salts thereof, wherein the process comprises the steps of:
(a) preparing 4-(1,2,4-triazol-1-yl-methyl)aniline hydrochloride (IX) by adding alcoholic HCl (preferably ethanolic HCl) to a solution of 4-(1,2,4-triazol-1-yl-methyl)aniline (II) in alcohol (preferably in methanol):

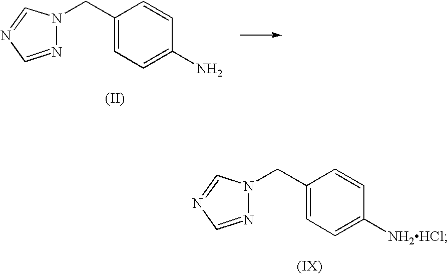

(b) diazotising 4-(1,2,4-triazol-1-yl-methyl)aniline hydrochloride (IX) using an excess of sodium nitrite (preferably up to 7.0 eq.) in the presence of an acid at a low temperature (preferably between −5 to 0° C.), followed by reduction with an excess of sodium sulfite (preferably up to 6.0 eq.) at a temperature in the range of 25-70° C. (preferably between 65-70° C.) for about 1 hour to obtain hydrazine derivative (IV):

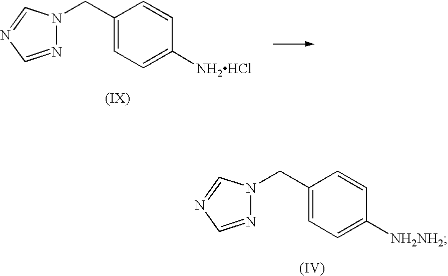

(c) adding hydrochloric acid or sulfuric acid to the reaction mass at a temperature in the range of 65-70° C., maintaining the reaction mass at 65-70° C. for about 1 hour, and then cooling the reaction mass to 25-30° C.;
(d) adding 4-N,N-dimethylamino-butyraldehyde diethyl acetal (X) to the reaction mass, and maintaining the reaction temperature below 30° C. (preferably at a temperature in the range of 25-30° C.) for about 24 hours:

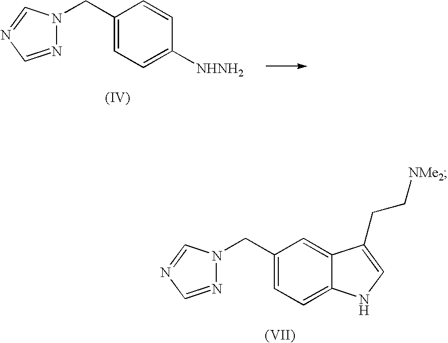

(e) extracting and purifying the rizatriptan free base (VII); and
(d) optionally converting the rizatriptan free base (VII) into a pharmaceutically acceptable salt thereof.

It is preferred to prepare and isolate the 4-(1,2,4-triazol-1-yl-methyl)aniline hydrochloride (IX) and then subject it to diazotisation. It is observed that diazotisation of 4-(1,2,4-triazol-1-yl-methyl)aniline hydrochloride (IX) with an excess of sodium nitrite followed by reduction with an excess of sodium sulfite results in complete formation of hydrazine derivative (IV) with better purity compared to a similar conversion starting with 4-(1,2,4-triazol-1-yl-methyl)aniline (II).

In step (c), conc. hydrochloric acid or conc. sulfuric acid is preferably added to a solution of hydrazine derivative (IV). Preferably the reaction mass is maintained at temperatures between 25-70° C., preferably between 65-70° C., for about 1 hour, followed by cooling to 20-30° C.

In step (d), it is a preferred embodiment of this invention to add 4-N,N-dimethylamino-butyraldehyde diethyl acetal (X) to the reaction mass whilst maintaining the reaction temperature below 30° C. and to maintain the reaction mass at temperatures between 25-90° C., preferably between 25-30° C., until completion of the reaction. It was found that indolyzation between 25-30° C. for approximately 24 hours minimizes formation of dimer impurities.

The preferred work-up is basification of the reaction mass with a suitable base such as aqueous ammonia, followed by extraction of rizatriptan base into ethyl acetate. It was observed that ethyl acetate is the solvent of choice. Ethyl acetate extracts contain a major proportion of rizatriptan base along with a relatively small proportion of impurities. On the contrary, it was observed that methylene chloride extracts contain rizatriptan base along with a relatively large proportion of impurities.

In yet another embodiment of the present invention, the preferred way to purify the rizatriptan base is via its succinate salt. To the combined ethyl acetate extracts, an aqueous solution of succinic acid is added. The rizatriptan succinate formed remains in the aqueous layer. This leaves the impurities in the ethyl acetate. It also avoids distillation of ethyl acetate to isolate rizatriptan, which is time consuming. Washing of the aqueous solution of rizatriptan succinate with ethyl acetate further assures complete removal of impurities. Basification of the aqueous solution of rizatriptan succinate with a base such as aqueous sodium hydroxide gives rizatriptan base with high purity.

Rizatriptan benzoate salt can be prepared by following the procedure described in the prior art. Dissolution of pure rizatriptan base (either in the form of an oil or solid) in ethanol and addition of a solution of benzoic acid in tert-butyl methyl ether (TBME), filtration, and then crystallization of the rizatriptan benzoate with ethanol gives rizatriptan benzoate (I) with high purity.

Rizatriptan base obtained as per the present invention gives rizatriptan benzoate as a free flowing solid, whereas it was observed that the rizatriptan base obtained by following the procedures described in the prior art gives a sticky solid which is difficult to handle on large scale.

The process disclosed in this application is capable of providing rizatriptan base and pharmaceutically acceptable salts thereof in high purity consistently irrespective of the scale of preparation.

The present invention further provides a pharmaceutical composition comprising rizatriptan, or a pharmaceutically acceptable salt thereof, preferably the benzoate, succinate or fumarate salt, which have been prepared in accordance with any of the above aspects of the invention. It also provides the use of the aforesaid pharmaceutical compositions for the preparation of a medicament for the treatment of migraine.

The dosage form can be a solution or suspension form, but is preferably solid and comprises one or more conventional pharmaceutically acceptable excipient(s). Preferred dosage forms in accordance with the invention include tablets, capsules and the like. Tablets can be prepared by conventional techniques, including direct compression, wet granulation and dry granulation. Capsules are generally formed from a gelatine material and can include a conventionally prepared granulate of excipients and adduct or solvate in accordance with the invention.

The following paragraphs enumerated consecutively from 1 through 41 provide for various aspects of the present invention. In one embodiment, the present invention provides:
1. A process for the preparation of rizatriptan (VII) or a pharmaceutically acceptable salt thereof, comprising the steps of:
(a) diazotising 4-(1,2,4-triazol-1-yl-methyl)aniline (II) or a salt thereof and then reducing the diazotised product to form hydrazine (IV):

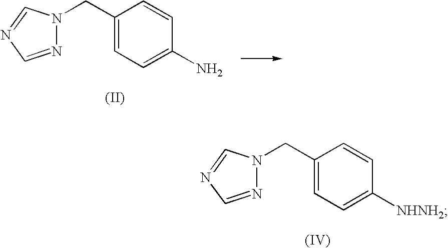

(b) reacting hydrazine (IV), with or without isolation, with 4-N,N-dimethylamino-butyraldehyde or a carbonyl-protected form thereof to form rizatriptan free base (VII):

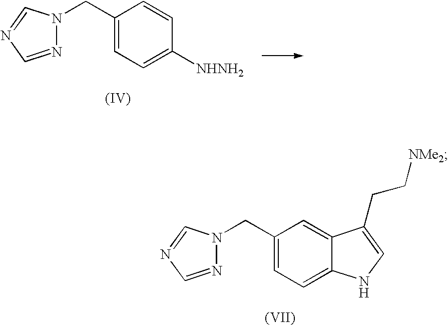

(c) extracting and purifying the rizatriptan free base (VII); and
(d) optionally converting the rizatriptan free base (VII) into a pharmaceutically acceptable salt thereof.
2. A process according to paragraph 1, wherein the salt of aniline (II) used in step (a) is a mineral acid salt or an organic carboxylic acid salt.
3. A process according to paragraph 2, wherein the mineral acid salt is the hydrochloric, hydrobromic or sulfuric acid salt.
4. A process according to paragraph 3, wherein the mineral acid salt is the hydrochloric acid salt (IX) obtained by adding alcoholic HCl to a solution of 4-(1,2,4-triazol-1-yl-methyl)aniline (II) in the presence of an alcohol.
5. A process according to paragraph 4, wherein the hydrochloric acid salt (IX) is obtained by adding ethanolic HCl to a solution of 4-(1,2,4-triazol-1-yl-methyl)aniline (II) in the presence of methanol.
6. A process according to paragraph 2, wherein the organic carboxylic acid salt is the formic, acetic, benzoic or sulfonic acid salt.
7. A process according to paragraph 6, wherein the sulfonic acid salt is the benzene sulfonic or toluene sulfonic acid salt.
b 8. A process according to any one of paragraphs 1 to 7, wherein the reduction in step (a) is carried out using sodium sulfite or sodium dithionite.
9. A process according to any one of paragraphs 1 to 8, wherein the carbonyl-protected form of 4-N,N-dimethylamino-butyraldehyde used in step (b) is the diethyl acetal.
10. A process according to any one of paragraphs 1 to 9, wherein step (b) is carried out in the presence of an acid.
11. A process according to paragraph 10, wherein the acid used in step (b) is sulfuric acid or hydrochloric acid.
12. A process according to any one of paragraphs 1 to 11, wherein the cyclization reaction of step (b) is carried out at a temperature of 25-30° C.
13. A process according to any one of paragraphs 1 to 12, wherein step (c) comprises the steps of: (c1) basifying the reaction mixture, (c2) extracting crude rizatriptan base into an organic solvent, (c3) extracting rizatriptan into an acidic aqueous solution, (c4) basifying the aqueous solution comprising acidic rizatriptan, (c5) re-extracting purified rizatriptan base into an organic solvent, and (c6) removing the organic solvent.
14. A process according to any one of paragraphs 1 to 13, wherein step (c) comprises the steps of: (cl) basic work-up at a pH of about 8.5-9, (c2) extraction of crude rizatriptan base into an organic solvent, (c3) purification by extraction into an aqueous solution of an organic acid,
(c4) liberation of purified rizatriptan base, (c5) re-extraction of the purified rizatriptan base into an organic solvent, and (c6) removal of the organic solvent.
15. A process according to paragraph 14, wherein in step (c1) aqueous ammonia is used.
16. A process according to paragraph 14 or 15, wherein in step (c2) crude rizatriptan base is extracted into ethyl acetate.
17. A process according to any one of paragraphs 14 to 16, wherein in step (c3) the organic acid used for extraction is a water soluble organic acid.
18. A process according to paragraph 17, wherein the water soluble organic acid is oxalic, citric or succinic acid.
19. A process according to paragraph 18, wherein the water soluble organic acid is succinic acid.
20. A process according to any one of paragraphs 14 to 19, wherein the aqueous solution of acidic rizatriptan obtained in step (c3) is washed with an organic solvent.
21. A process according to paragraph 20, wherein the organic solvent is ethyl acetate.
22. A process according to any one of paragraphs 14 to 21, wherein in step (c4) the aqueous solution of acidic rizatriptan is basified to a pH of about 8.5-9 with a base.
23. A process according to paragraph 22, wherein the base is aqueous sodium hydroxide.
24. A process according to any one of paragraphs 14 to 23, wherein in step (c5) the purified rizatriptan base is re-extracted into ethyl acetate.
25. A process according to any one of paragraphs 1 to 24, wherein the purified rizatriptan base obtained in step (c) is more than 99.7% pure (as measured by HPLC).
26. A process according to any one of paragraphs 1 to 25, wherein the purified rizatriptan base obtained in step (c) is practically free of dimeric and other impurities.
27. A process according to any one of paragraphs 1 to 26, wherein the purified rizatriptan base obtained in step (c) is practically free of dimer impurity (XI).
28. A process according to paragraph 27, wherein the dimer impurity (XI) is less than 0.05% (as measured by HPLC).
29. A process according to any one of paragraphs 1 to 28, wherein the purified rizatriptan base in step (c) is obtained on an industrial scale.
30. A process according to any one of paragraphs 1 to 29, wherein the purified rizatriptan base in step (c) is obtained from 4-(1,2,4-triazol-1-yl-methyl)aniline (II) or a salt thereof in a yield of 50% or more.
31. A process according to any one of paragraphs 1 to 30, wherein the pharmaceutically acceptable salt formed in step (d) is the benzoate, oxalate, succinate, hydrochloride, hydrobromide, acetate, propionate, maleate or fumarate salt.
32. A process according to any one of paragraphs 1 to 31, wherein the process is carried out without the use of column chromatography.
33. Rizatriptan or a pharmaceutically acceptable salt thereof, prepared by a process according to any one of paragraphs 1 to 32.
34. Rizatriptan or a pharmaceutically acceptable salt thereof, with more than 99.7% HPLC purity (as measured by HPLC).
35. Rizatriptan or a pharmaceutically acceptable salt thereof, practically free of dimeric and other impurities.
36. Rizatriptan or a pharmaceutically acceptable salt thereof, practically free of dimer impurity (XI).
37. Rizatriptan or a pharmaceutically acceptable salt thereof according to paragraph 36, wherein the dimer impurity (XI) is less than 0.05% (as measured by HPLC).
38. Rizatriptan according to any one of paragraphs 33 to 37, wherein the pharmaceutically acceptable salt is the benzoate, oxalate, succinate, hydrochloride, hydrobromide, acetate, propionate, maleate or fumarate salt.
39. A pharmaceutical composition comprising rizatriptan or a pharmaceutically acceptable salt thereof according to any one of paragraphs 33 to 38.
40. Use of a compound according to any one of paragraphs 33 to 38 or use of a composition according to paragraph 39, for the manufacture of a medicament for the treatment or prevention of migraine.
41. A method of treating or preventing migraine, comprising administering a compound according to any one of paragraphs 33 to 38 or a composition according to paragraph 39, to a patient in need thereof.

The details of the invention, its objects and advantages are explained hereunder in greater detail in the following non-limiting examples.

### Examples

### Example 1

### Conversion of 4-(1,2,4-triazol-1-yl-methyl)phenylamine (II) into the corresponding diazonium chloride salt (III)

To a solution of 4-(1,2,4-triazol-1-yl-methyl)phenylamine (II) (2.4 kg) in methanol (341), ethanolic HCl (4.81) was added at 5° C. The precipitated amine hydrochloride (IX) was filtered, washed with methanol (21) and dried. The amine hydrochloride (IX) was dissolved in water (8.751), and conc. HCl (11.51) was added at 25° C. The solution was cooled below −5° C. A solution of sodium nitrite (6.68 kg) in water (3.31) was added to the above solution whilst maintaining the temperature below −5° C. After completion of the addition, the mixture was stirred for 30 minutes resulting in formation of a diazonium chloride (III) solution.

### Example 2

### Conversion of 4-(1,2,4-triazol-1-yl-methyl)phenyl-diazonium chloride (III) into the corresponding hydrazine (IV)

The diazonium chloride (III) solution obtained in example 1 was rapidly added to a well-stirred solution of sodium sulfite (10 kg) in water (401) below 10° C. The mixture was stirred for 2 hours at 65-70° C. to obtain an aqueous solution of hydrazine (IV).

### Example 3

### Reaction of 4-(1,2,4-triazol-1-yl-methyl)phenyl-hydrazine (IV) with 4-N,N-dimethylamino-butyraldehyde diethyl acetal (X) (Fischer indole synthesis)

To the aqueous hydrazine (IV) solution obtained in example 2, conc. sulfuric acid (4.371) was added and the temperature of the reaction mixture was maintained for 2 hours at 65-70° C. After cooling to 20-25° C., 4-N,N-dimethylamino-butyraldehyde diethyl acetal (X) (3.15 kg) was added. The reaction was heated to 70° C. and maintained for 3-4 hours. After completion of the reaction, the reaction mixture was allowed to cool to 15-20° C. To this mixture, 25% aq. ammonia (7.251) was added to adjust the pH to 8.5-9. The solution was extracted with ethyl acetate (4×12.251). A solution of succinic acid (2.45 kg) in water (301) was added to the ethyl acetate extract. The mixture was stirred for 15 minutes. The aqueous layer was separated and washed with ethyl acetate (2×51). The aqueous layer was basified with 20% aq. NaOH to adjust the pH to 8.5-9. The solution was extracted with ethyl acetate (4 x 51). The combined ethyl acetate extracts were concentrated to give rizatriptan free base (VII) as oil (2.8 kg, 75.5% from 4-(1,2,4-triazol-1-yl-methyl)phenylamine (II)). Purity =99.7-99.9% (as measured by HPLC).

### Example 4

### Preparation of rizatriptan benzoate (I) from rizatriptan free base (VII)

To a solution of rizatriptan free base (VII) (2.8 kg) in ethanol (2.81) and tert-butyl methyl ether (5.61), a solution of benzoic acid (1.52 kg) in tert-butyl methyl ether (5.61) was added. The mixture was stirred for 1 hour. The sticky solid obtained was filtered. The sticky solid was given a slurry wash with acetone (8.751). The product obtained was filtered and dried to obtain crude product as solid (1.75 kg). The crude solid was crystallized from ethanol (10.51) to obtain pure rizatriptan benzoate (I) as off-white solid (1.0 kg, 24.6% from rizatriptan free base (VII)). Purity=99.7-99.9% (as measured by HPLC).

What is claimed is:

1. A process for the preparation of rizatriptan (VII) or a pharmaceutically acceptable salt thereof, comprising the steps of:
(a) diazotising 4-(1,2,4-triazol-1-yl-methyl)aniline (II) or a salt thereof and then reducing the diazotised product to form hydrazine (IV): 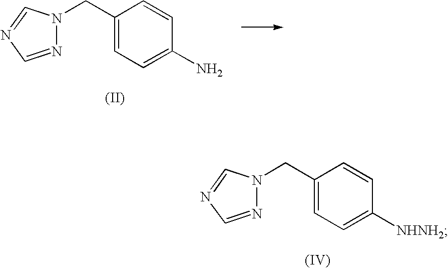
(b) reacting hydrazine (IV), with or without isolation, with 4-N,N-dimethylamino-butyraldehyde or a carbonyl-protected form thereof to form rizatriptan free base (VII): 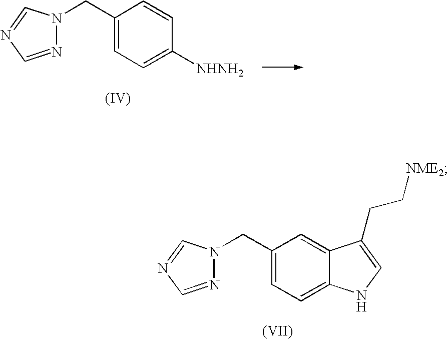
(c) extracting and purifying the rizatriptan free base (VII); and
(d) optionally converting the rizatriptan free base (VII) into a pharmaceutically acceptable salt thereof.

2. A process according to claim 1, wherein the salt of aniline (II) used in step (a) is:
(i) a mineral acid salt or an organic carboxylic acid salt; and/or
(ii) a hydrochloric, hydrobromic or sulfuric acid salt; and/or
(iii) a hydrochloric acid salt (IX) obtained by adding alcoholic HCl to a solution of 4-(1,2,4-triazol-1-yl-methyl)aniline (II) in the presence of an alcohol; and/or
(iv) a hydrochloric acid salt (IX) obtained by adding ethanolic HCl to a solution of 4-(1,2,4-triazol-1-yl-methyl)aniline (II) in the presence of methanol; and/or
(v) a formic, acetic, benzoic or sulfonic acid salt; and/or
(vi) a benzene sulfonic or toluene sulfonic acid salt.

3. A process according to claim 1, wherein:
(i) the reduction in step (a) is carried out using sodium sulfite or sodium dithionite; and/or
(ii) the carbonyl-protected form of 4-N,N-dimethylamino-butyraldehyde used in step (b) is the diethyl acetal; and/or
(iii) step (b) is carried out in the presence of an acid; and/or
(iv) step (b) is carried out in the presence of an acid, wherein the acid is sulfuric acid or hydrochloric acid; and/or
(v) the cyclization reaction of step (b) is carried out at a temperature of 25-30° C.; and/or
(vi) step (c) comprises the steps of: (c1) basifying the reaction mixture, (c2) extracting crude rizatriptan base into an organic solvent, (c3) extracting rizatriptan into an acidic aqueous solution, (c4) basifying the aqueous solution comprising acidic rizatriptan, (c5) re-extracting purified rizatriptan base into an organic solvent, and (c6) removing the organic solvent.

4. A process according to claim 1, wherein step (c) comprises the steps of: (c1) basic work-up at a pH of about 8.5-9, (c2) extraction of crude rizatriptan base into an organic solvent, (c3) purification by extraction into an aqueous solution of an organic acid, (c4) liberation of purified rizatriptan base, (c5) re-extraction of the purified rizatriptan base into an organic solvent, and (c6) removal of the organic solvent.

5. A process according to claim 4, wherein:
(i) in step (c1) aqueous ammonia is used; and/or
(ii) in step (c2) crude rizatriptan base is extracted into ethyl acetate; and/or
(iii) in step (c3) the organic acid used for extraction is a water soluble organic acid; and/or
(iv) in step (c3) the organic acid used for extraction is oxalic, citric or succinic acid; and/or
(v) in step (c3) the organic acid used for extraction is succinic acid; and/or
(vi) the aqueous solution of acidic rizatriptan obtained in step (c3) is washed with an organic solvent; and/or
(vii) the aqueous solution of acidic rizatriptan obtained in step (c3) is washed with ethyl acetate; and/or
(viii) in step (c4) the aqueous solution of acidic rizatriptan is basified to a pH of about 8.5-9 with a base; and/or
(ix) in step (c4) the aqueous solution of acidic rizatriptan is basified to a pH of about 8.5-9 with aqueous sodium hydroxide; and/or
(x) in step (c5) the purified rizatriptan base is re-extracted into ethyl acetate.

6. A process according to claim 1, wherein the purified rizatriptan base obtained in step (c) is:
(i) more than 99.7% pure (as measured by HPLC); and/or
(ii) practically free of dimeric and other impurities; and/or
(iii) practically free of dimer impurity (XI); and/or
(iv) practically free of dimer impurity (XI), wherein the dimer impurity (XI) is less than 0.05% (as measured by HPLC); and/or
(v) obtained on an industrial scale; and/or
(vi) obtained from 4-(1,2,4-triazol-1-yl-methyl)aniline (II) or a salt thereof in a yield of 50% or more.

7. A process according to claim 1, wherein the pharmaceutically acceptable salt formed in step (d) is the benzoate, oxalate, succinate, hydrochloride, hydrobromide, acetate, propionate, maleate or fumarate salt.

8. A process according to claim 1, wherein the process is carried out without the use of column chromatography.

9. Rizatriptan or a pharmaceutically acceptable salt thereof, prepared by a process according to claim 1.

10. Rizatriptan or a pharmaceutically acceptable salt thereof:
(i) with more than 99.7% HPLC purity (as measured by HPLC); and/or
(ii) practically free of dimeric and other impurities; and/or
(iii) practically free of dimer impurity (XI); and/or
(iv) practically free of dimer impurity (XI), wherein the dimer impurity (XI) is less than 0.05% (as measured by HPLC).

11. Rizatriptan according to claim 9, wherein the pharmaceutically acceptable salt is the benzoate, oxalate, succinate, hydrochloride, hydrobromide, acetate, propionate, maleate or fumarate salt.

12. Rizatriptan according to claim 10, wherein the pharmaceutically acceptable salt is the benzoate, oxalate, succinate, hydrochloride, hydrobromide, acetate, propionate, maleate or fumarate salt.

13. A pharmaceutical composition comprising rizatriptan or a pharmaceutically acceptable salt thereof according to claim 9.

14. A pharmaceutical composition comprising rizatriptan or a pharmaceutically acceptable salt thereof according to claim 10.

15. A method of treating or preventing migraine, comprising administering a compound according to claim 9, to a patient in need thereof.

16. A method of treating or preventing migraine, comprising administering a compound according to claim 10, to a patient in need thereof.
